(19) United States
(12) Patent Application Publication
(10) Pub. No.: US 2025/0235301 A1
(43) Pub. Date: Jul. 24, 2025

(54) EMBRYO COLLECTION DEVICE FOR COLLECTING EMBRYOS IN THE UTERINE HORN OF A SOW OR A GILT

(71) Applicant: Norsvin SA, HAMAR (NO)

(72) Inventors: Bjørn GULBRANDSEN, HAMAR (NO); Reina JOCHEMS, HAMAR (NO); Marianne OROPEZA-MOE, SANDNES (NO); Silje Katrine NES, SANDNES (NO); Louisa Jane ZAK, BEUNINGEN (NL)

(73) Assignee: Norsvin SA, HAMAR (NO)

(21) Appl. No.: 18/705,245

(22) PCT Filed: Nov. 11, 2022
(86) PCT No.: PCT/EP2022/081664

§ 371 Date: Apr. 26, 2024

(30) Foreign Application Priority Data
Nov. 11, 2021 (NL) 2029705

(51) Int. Cl. A61D 19/04

(52) U.S. Cl. CPC A61D 19/04

(57) ABSTRACT

An embryo collection device for collecting embryos in the uterine horn of a pig, in particular a sow or a gilt, includes an introduction probe to be partially introduced into the cervix of the pig, provided with a guide lumen; and a flushing catheter, slidably arranged in the guide lumen, including: a proximal and a distal end, an inflation lumen and a flushing lumen, an inflatable cuff fluidly connected to the inflation lumen to be inflated for at least partially closing the uterine horn towards a proximal end thereof; and a flushing section between the inflatable cuff and the distal end to be introduced inside the uterine horn, including a flushing opening fluidly connected to the flushing lumen; and a tip element arranged at the distal end, shaped to steer the flushing catheter, for example such that upon insertion of the introduction probe in the cervix, the flushing catheter may be advanced further into the reproductive tract passing the cervix and uterine body and entering the uterine horn for flushing the uterine horn through the flushing opening.

### FIELD OF THE INVENTION

The present invention relates to an embryo collection device for collecting embryos in the uterine horn of a pig, an embryo collection assembly, and to a method for flushing embryos.

### BACKGROUND OF THE INVENTION

Due to the anatomical complex structure of the reproductive tract of a pig, collection of embryos is relatively difficult. In particular, advancement of a catheter in the cervix and the uterine horns remained a challenging task. Amongst others, complicating factors are the relatively narrow openings of the cervix, the cervical folds, the relatively sharp bend of approximately 90 degrees ventrally near the uterine horns, and, in particular, the length of the uterine horns and their tortuous shape.

Depending on the background of the specific pig, such as breeding method, breed of pig and possible undergone surgery, a different collection procedure and/or a different type of catheter may have been required.

As a result, conventional catheter systems have not been applied in sows and/or gilts efficiently and obtained results in terms of collected embryos and quality of the embryos were relatively low. Also, multiple attempts were usually necessary before few embryos could be collected. Further, anaesthesia was often required.

As an alternative, a surgical procedure may be performed to collect embryos. However, surgery requires special medical expertise and may involve health risks to the pig.

### OBJECT OF THE INVENTION

It is therefore an object of the invention to provide an embryo collection device that overcomes the disadvantages of the prior art, or at least to provide an alternative embryo collection device, for example an embryo collection device which enables to collect embryos more efficiently and with increased wellbeing of the pig.

### DETAILED DESCRIPTION

The present invention provides an embryo collection device for collecting embryos in the uterine horn of a pig, in particular a sow or a gilt. The embryo collection device comprises an introduction probe to be partially introduced into the cervix of the pig, provided with a guide lumen; and a flushing catheter, slidably arranged in the guide lumen.

The flushing catheter comprises a proximal end and a distal end, an inflation lumen extending from an inflation port at the proximal end and a flushing lumen extending from an flushing port at the proximal end, an inflatable cuff fluidly connected to the inflation lumen to be inflated for at least partially closing the uterine horn at a proximal end thereof and a flushing section arranged between the inflatable cuff and the distal end to be introduced inside the uterine horn, comprising a flushing opening fluidly connected to the flushing lumen.

The flushing section comprises a tip element arranged at the distal end. The tip element is shaped to steer the flushing catheter, for example such that upon insertion of the introduction probe in the cervix, the flushing catheter may be advanced further into the reproductive tract passing the cervix and uterine body and entering the uterine horn for flushing the uterine horn through the flushing opening.

The introduction probe may, with a distal end thereof, be configured to be positioned in the cervix of the pig to introduce the flushing catheter in the cervix. The introduction probe may, towards the distal end, have a decreasing diameter. An end surface of the introduction probe at the distal end may be rounded. A rounded end and/or a decreasing diameter may assist to gradually find a way through the caudal opening and/or along the cervical folds.

The introduction probe may be configured to be positioned, from the perspective of an operator, in front of the caudal opening of the cervix. The introduction probe may be relatively stiff to allow an operator to feel the position of the introduction probe with respect to the tissue of the cervix, such that the introduction probe may be inserted partially into the cervix, for example through the caudal opening but not through the cranial opening thereof.

Therewith, the introduction probe may provide external support to the flushing catheter in the cervix, such that excessive bending of the flushing catheter can be avoided. Furthermore, the introduction probe provides space through which the flushing catheter may be advanced relatively easily compared to advancing a catheter without introduction probe, for example a catheter having a guide wire. It has been found that partial insertion of the introduction probe is sufficient for guiding the flushing catheter through the cervix. Further, by not having to pass through the cranial opening with the introduction probe, the procedure may be performed relatively fast and the risk of damaging the uterus with the introduction probe is reduced.

The flushing catheter is slidably arranged in the guide lumen of the introduction probe and is configured to be introduced in the cervix with the introduction probe. Upon insertion of the introduction probe, the flushing catheter may be advanced to extend out of the introduction probe into the cervix and/or the uterus of the pig. The flushing catheter may for example be configured to extend through an opening in communication with the guide lumen at the distal end of the introduction probe.

The guide lumen may follow the shape of the introduction probe. The guide lumen may be relatively straight. The guide lumen may be provided with a smooth inner surface. An inner diameter of the guide lumen may be larger than a diameter of narrow passages in the cervix, e.g. larger than a distance between side walls of the cervix in a natural state. These features have been found to ease sliding of the flushing catheter through the guide lumen.

The flushing catheter comprises a tip element shaped to steer the catheter, such that the flushing catheter may pass the cervix and uterine body into an uterine horn. In particular, the flushing catheter may pass the cervix, uterine body and/or uterine horn without being supported by the introduction tube through the entire cervix.

The flushing catheter may be configured to be advanced through the cranial opening of the cervix when the introduction tube is inserted partially into the cervix and not through the cranial opening of the cervix.

The advantageous combination of the introduction probe and flushing catheter allows for an efficient introduction in the reproductive tract for advancing the flushing catheter tip into the uterine horn for flushing. By introducing the flushing catheter with the introduction probe, the cervix may be passed in a relatively short time. It has been found that with the embryo collection device, relatively high cervical pass ratios may be achieved of up to 100% in a single attempt. As such, the procedure can be performed relatively quickly and is relatively advantageous for animal welfare.

The tip element may be advanced into a selected uterine horn and may be configured to steer the flushing catheter into and through the selected uterine horn.

The flushing catheter may be relatively torsion stiff such that the tip element may be rotated by rotating a proximal end of the catheter, such that a uterine horn may be selected by rotation of the tip element.

As the tip element is shaped to steer the flushing catheter, a relatively good manoeuvrability may be achieved without guide wire. Therewith, a guide wire or other support element for constricting movement freedom of the tip element in the uterine horn may be omitted.

Upon advancing the flushing catheter into the uterine horn towards a desired depth, the inflatable cuff may be inflated via the inflation lumen to close a part of the uterine horn at least partially, after which flushing of the closed-off part may be performed via the flushing section. The uterine horn may, for example, be at least partially closed at a proximal end thereof to perform flushing of substantially the entire uterine horn, or alternatively, the uterine horn may be closed partially to perform flushing of a smaller part of the uterine horn.

The inflatable cuff may partially or completely close the uterine horn. This way, a relatively high fluid recovery may be achieved, for example at least 50% or at least 70%.

In particular, the flushing catheter may be positioned to perform flushing at a desired depth in the uterine horn, for example deep in the uterine horn. The flushing catheter may be advanced in the uterine horn up to a greater or lesser depth. Therefore, the flushing catheter may be used in multiple circumstances, for example when the pig has abnormalities in the reproductive tract, when the pig has undergone surgery, and/or has differences in the reproductive tract due to aging or earlier litter(s) of the pig. This allows the embryo collection device to be used for different pig breeds, in different countries and according to different breeding methods.

Having a relatively good manoeuvrability, a relatively good animal welfare can be achieved with less or possibly even completely without any anaesthetic, such that animal wellbeing can potentially be improved, and the use of medication can therefore potentially be reduced.

Therewith, the flushing of embryos can be carried out without substantial health risks to the pig. As a result, even no signs of discomfort were observed during in vivo tests.

By collecting embryos, pregnancy can be prevented and/or the chances can at least be reduced. Further embryos may be collected for pig breeding and/or for scientific research.

In an embodiment, the tip element is a rounded cone. The tip element may be an oblique cone having a base plane and a top. The cone may have a rounded top.

In an embodiment, the tip element is a bent rounded cone. The tip element may be bent having the cone axis in a first biasing direction. The second biasing direction may coincide with the first biasing direction. The tip element may be bent in the second biasing direction at an angle that is less than 90°, in particular less than 25°, for example 5°-10° with respect to a longitudinal axis of the flushing section.

The top may also be biased in a second biasing direction, such that a projection of the top on the base plane is not located in the centre of the plane.

As such, the tip element is biased in the first and/or second biasing direction and, when advanced against a transverse surface, the flushing catheter is steered in the respective biasing direction, such that by orienting the tip element around its longitudinal axis, a position of the rounded cone with respect to uterine horns may be selected. This way, the first and/or second biasing direction may be chosen to coincide with one of the uterine horns.

Relatively convenient steerability of the flushing catheter may achieved by having a relatively high torsional stiffness to allow for rotation around a longitudinal axis, and a relatively low bending stiffness for following the shape of the uterine horn. Thereby, the introduction tube may prevent early bending of the flushing catheter with the guide lumen.

Pushability of the flushing catheter may be ensured by having a relatively high longitudinal stiffness to be advanced through the cranial opening of the cervix and into the uterine horn.

The inflatable cuff may be arranged on a fixed distance to the tip element, such that a relatively constant length of the uterine horn may be flushed. Flushing a constant length may allow to use a relatively constant volume of flushing fluid.

Varying the distance and/or volume of flushing with flushing procedure could lead to varying results in the number of embryos collected, due to the fact that the flushing may then be performed on different parts of the uterine horn or with varying intensity, respectively.

In an embodiment, the flushing section comprises an elongate element that spaces the inflatable cuff from the tip element along a longitudinal axis, for example wherein a length from the distal end to the inflatable cuff is 5-70 cm, for example 20-55 cm. By having a larger distance, a larger part of the uterine horn may be flushed. However, by having a smaller distance, the flushing catheter may be employed in a larger variety of uterine horns, while still being able to at least partially close the uterine horn.

Additionally, it has been found that flushing a part of the uterine horn deep in the uterine horn yields highest number of collected embryos. A distance of 30-40 cm has been found to provide the highest yield.

Flushing of the at least partially closed part of the uterine horn may be performed by providing a flushing fluid via the flushing lumen and the flushing opening. The flushing opening may have a diameter of equal to a diameter of the flushing lumen, or another diameter providing for gradual widening or narrowing of a flushing fluid flow.

A diameter of the flushing lumen may for example be 2-6 mm, for example 2.5 mm or 4 mm.

The flushing fluid may be a liquid, such as a buffered saline solution. The flushing fluid may comprise an embryo culture medium.

In an embodiment, the flushing opening is arranged in the elongate element and/or in the tip element. By providing the flushing fluid nearby the distal end of the flushing catheter, flushing may be performed deep in the uterine horn.

In an embodiment, the tip element is less rigid than the elongate element. This way, the chances of the flushing catheter getting stuck in the reproductive tract may be reduced due to elasticity of the tip element. Further, a relatively flexible tip element allows the tip element to bend into curves for advantageous steering of the flushing catheter

The inflatable cuff may be inflated between an empty state, wherein the inflatable cuff is compact, and an inflated state, wherein the inflatable cuff reaches larger outer dimensions. The inflatable cuff may have outer dimensions that, in an inflated state, substantially correspond to inner dimensions of a uterine horn to at least partially close the uterine horn for flushing fluid and/or for embryos. The inflatable cuff may have an inflated volume of at least 10 mL in the inflated state, for example at least 50 mL.

In an embodiment, a length of the flushing catheter from the proximal end up to the inflatable cuff is at least 100 cm, for example at least 150 cm, in particular 200 to 350 cm. This length has been found to allow flushing deep in the uterine horn.

An outer diameter of the flushing catheter may be at least 4 mm, for example 8 or 10 mm. This outer diameter has been found to allow for sufficient torsional and longitudinal stiffness for sufficient steerability and pushability of the catheter, while still providing sufficient bendability to follow the uterine horns.

In an embodiment, the tip element is autoclave-resistant. In addition to the tip element, the flushing section, the flushing catheter and/or the introduction probe may partially or entirely be autoclave-resistant. The tip element may be autoclave-resistant for at least 15, such as 30, minutes at 121° C., and/or at least 3, such as 6, minutes at 134° C., for example at a steam pressure of 2-5 bar. By having autoclave-resistance, safe reuse of the catheter and/or introduction probe may be possible.

The tip element, the flushing section and/or the flushing lumen may have a smooth surface. By having a smooth surface, adhesion of embryos on the surface may be reduced, such that the chances of embryo damaged on an outer surface are lowered.

In an embodiment, the flushing catheter may partially or entirely be made of a silicone material, such as silicone rubber. Silicone rubber has been found not to be toxic for the embryos.

The tip element may be made from 3d-printed silicone rubber, for example silicone rubber having an elastic modulus of 2.5-7.5 N/mm², such as TrueSil A50, which provides flexibility for advancement through the uterine horns.

In an embodiment, the tip element is connected at the distal end via a glue, in particular a silicone glue, such as Dow Corning 782.

In an embodiment the flushing catheter comprises a collection lumen extending from an embryo collection port at the proximal end towards the flushing section, and the flushing section comprises a collection opening fluidly connected to the collection lumen.

This way, inflow of flushing fluid through the flushing lumen may be provided separately from, and possibly simultaneous with, the collection of flushing fluid and embryos via the collection lumen. As such, flushing fluid may be provided while embryos are collected such that flushing may be performed during a comparatively longer period.

The collection lumen may have similar properties and dimensions as the flushing lumen. Alternatively, the collection lumen may have a larger diameter than the flushing lumen to improve flow of collected fluid and embryos through the collection lumen.

In an embodiment, the flushing opening and the collection opening are spaced at a distance from each other, in particular wherein the collection opening is spaced towards the inflatable cuff, and wherein the flushing opening is spaced towards the tip element. In particular, the flushing opening may be located in the tip element such that flushing fluid can be provided near the distal end.

Multiple flushing openings and/or multiple collection openings may be arranged on the flushing section, for example evenly distributed over the flushing section or grouped together, e.g. spaced at a distance from each other as described above.

The flushing openings and/or the collection openings may be spaced evenly around the circumference at a respective cross section of the flushing section, such that even flushing of the uterine horn, respectively collection, may be possible.

The introduction probe may comprise a proximal end and a distal end and extend therebetween. The proximal end of the introduction probe may comprise a manipulator and/or a grip for manipulation by an operator.

In addition or alternatively, the proximal end of the flushing catheter may comprise a manipulator and/or a grip for manipulation by an operator.

The distal end may comprise a catheter opening through which the flushing catheter may be advanced to extend out of the catheter opening, for example into the cervix and/or the uterus of the pig.

The introduction probe may be narrowing towards the distal end thereof. Additionally, or alternatively, the introduction probe may have a rounded and/or smooth shape towards the distal end. These features may ease introduction of the introduction probe.

In an embodiment the introduction probe is straight. A straight introduction probe may help an operator to introduce the introduction probe in the reproductive tract, in particular to move the introduction probe through the caudal opening and past the cervical folds.

In an embodiment the introduction probe has a flat outer surface. The outer surface may be a smooth surface. By having a flat and/or smooth outer surface, friction forces between the outer surface and the reproductive tract may be reduced.

A length of the introduction probe may be at least 40 cm, for example 50 cm. This way, the proximal end of the introduction probe may be manipulated while the distal end is introduced into the cervix, in particular through the caudal opening but not through the cranial opening thereof.

The introduction probe may be relatively rigid, such that the anatomy of the reproductive tract may be felt by the operator. In particular, the introduction tube may have a higher stiffness than the flushing catheter, such as a higher bending stiffness and a higher longitudinal stiffness.

In an embodiment the introduction probe comprises a holding element to hold the flushing catheter during introduction of the introduction probe. The holding element may hold the flushing catheter at a fixed position in the guide lumen. The holding element may be provided nearby the distal end of the introduction probe, for example in or adjacent to the guide lumen.

In an embodiment, the tip element and/or the flushing section may be connectable to the rest of the catheter having the inflatable cuff via a connection. The connection may be a threaded connection, friction connection, snap fit connection, other types of connection or combinations thereof. By having a connection, the flushing section and/or the rest of the catheter having the inflatable cuff may be manufactured separately. The connector may enable usage of commonly available catheter parts to be connected to the tip element and/or the flushing section to ease production of the flushing catheter. Additionally or alternatively, the connector may allow the catheter to be adapted to the specific animal.

In an embodiment, the connection of the tip element and/or the flushing section may be detachable. This way, a single catheter may be adapted to a specific animal.

Additional or alternative connections may be arranged between other parts of the catheter, e.g. by having connectors arranged therebetween.

The flushing section may comprise a connector, such as a screw thread connection, friction connector, snap fit connector, other connector or combinations thereof. A complementary connector adapted to cooperate with the connector of the flushing section may be arranged on the rest of the catheter having the inflatable cuff and/or on additional elongate elements. A commonly available catheter part may be provided with the complementary connector, or the complementary connector may be arranged therein, e.g. by milling.

The connector may comprise radial protrusions that are non-rotationally symmetric This way, rotation forces may be transferred via the connector. As such, rotation between the connector and a complementary connector may be reduced or avoided to improve steering of the flushing catheter. For example, the radial protrusions may provide the connector with a non-circular cross sectional shape, such as an oval shape.

The connector may comprise an indent having a first diameter and one or more ridges having a second diameter larger than the first diameter. The ridges may provide additional outer surface to the connector which may improve connection strength, e.g. by having a larger surface for friction and/or glue. Additionally or alternatively, the connector may connect by having a complementary connector snapping around the indent behind a ridge, or vice versa.

The invention further provides an embryo collection assembly, comprising an embryo collection device according to an embodiment; a flushing pump fluidly connectable to the flushing lumen via one of the at least two ports for pumping flushing fluid through the flushing opening; an inflation pump fluidly connectable to the inflation lumen via one of the at least two ports for inflating the inflatable cuff; and an embryo collection container, fluidly connectable to one of the at least two lumens for collecting embryos flushed out of the uterine horn.

The assembly allows the collection of embryos with benefits similar to those described hereinabove.

In an embodiment, the embryo collection assembly comprises an imaging system, such as an ultrasound or pulsating doppler system, for imaging a position of the tip element with respect to the uterine horn. Using an imaging system, the position of the tip element may be determined relatively precisely such that flushing may be performed in a specific part of the uterine horn. In particular, the position of the tip element deep in the uterine horn may be verified.

The invention further provides a use of an embryo collection device according to an embodiment of the invention and/or an embryo collection assembly according to an embodiment of the invention for collecting embryos in the uterine horn of a sow or a gilt.

The use provides benefits similar to those described hereinabove, and may particularly be useful for collection of embryos in sows and/or gilts.

In an embodiment, the introduction probe is introduced into the cervix but not advanced through the entire cervix, for example introduced up to ⅔ of the length of the cervix.

It has been found that partial insertion of the introduction probe is sufficient for guiding the flushing catheter through the cervix. Further, by not having to pass through the cranial opening with the introduction probe, the procedure may be performed relatively fast and the risk of damaging the uterus with the introduction probe is reduced.

In particular, the introduction probe may be introduced through the caudal opening but not through the cranial opening of the cervix.

In an embodiment, the tip element is advanced up to at least 50% of the length of the uterine horn, for example up to the tip of the uterine horn. This depth has been found to yield relatively high flushing yields.

In an embodiment, the embryo collection device is inserted for approximately 50 cm into the reproductive tract, upon which the flushing catheter is further advanced into the uterus, in particular at least 50 cm.

In an embodiment, upon insertion of the introduction probe in the cervix, the flushing catheter is advanced further into the reproductive tract passing the cervix and uterine body, upon which the tip element is rotated to select a uterine horn, upon which the catheter is advanced further to enter the selected uterine horn for flushing the uterine horn through the flushing opening.

In an embodiment, at least 40% of the flushing volume of flushing fluid is recovered. In particular 50-70% of the flushing volume may be recovered. Thus, according to the invention, a relatively high recovery ratio may be obtained, which may be much better than known techniques which typically result in relatively low recovery rates. For example, a flushing volume of at least 0,25 L is used, in particular 0,5 L.

The cervix may be passed in a relatively short time, for example within 20-30 seconds. In particular, the introduction tube may be positioned in front of the cranial opening within 20-30 seconds.

In an embodiment, the use comprises the step of determining a position of the tip element with respect to the uterine horn, in particular with respect to the tip of the uterine horn.

The position may be determined using an imaging system, such as an ultrasound or x-ray system. Doppler or pulsed doppler ultrasound may be used. The imaging system may comprise a rectal probe.

In an embodiment, flushing is performed 3-9 days after observing a standing reflex of the sow, in particular on day 5-7. This way, embryos may be collected relatively early. In particular, blastocysts may be collected. At this stage, the embryos may be located relatively deep in the uterine horn.

In an embodiment, flushing is performed using an embryo collection device having a length from the distal end to the inflatable cuff of 30-40 cm. Experiments indicate that at this length, most embryos may be found, for example most embryos on day 5 post oestrus.

In an embodiment, the use comprises the step of determining the presence and/or properties of the embryos, in particular blastocysts, e.g. by imaging the ovaries of the pig using an imaging system. Ovulation may for example be detected by imaging the ovaries with ultrasound.

As an example, a relatively high number of 5-20 embryos, in particular blastocysts, may be collected. Due to the high level of animal welfare, the embryos may be collected repeatedly, for example after 21 days.

In an embodiment, the use of the embryo collection device comprises a step of at least partial sterilisation in an autoclave.

The invention further provides a method for flushing a reproductive tract using an embryo collection device, for example according to any of the claims 1-10 and/or an embryo collection assembly, for example according to any of the claims 11-12, comprising the steps of: moving an introduction probe having a guide lumen partially into the reproductive tract-shaped object in a movement direction; moving a flushing catheter arranged in the guide lumen to slide within the guide lumen in the movement direction; rotating the flushing catheter with respect to the introduction probe to steer the flushing catheter with a tip element thereof in a direction angled to the movement direction; further moving the flushing catheter at a proximal end in the movement direction; inflating an inflatable cuff; providing flushing fluid into the flushing lumen; and collecting flushing fluid from the flushing lumen and/or from a collection lumen.

### BRIEF DESCRIPTION OF THE DRAWINGS

Further characteristics and advantages of the invention will now be elucidated by a description of embodiments of the invention, with reference to the accompanying drawings, in which:

FIG. 1 schematically depicts an embryo collection device according to an embodiment of the invention;

FIG. 2A schematically depicts a cross section of the embodiment of the embryo collection device of FIG. 1, along line A-A of FIG. 1;

FIG. 2B schematically depicts a cross section of the embodiment of the embryo collection device of FIG. 1, along line B-B of FIG. 1;

FIG. 3A schematically depicts a top view of a flushing section of the embryo collection device according to the embodiment of FIG. 1;

FIG. 3B schematically depicts a side view of a flushing section of an embryo collection device according to the embodiment of FIG. 1, wherein several dimensions are shown as an example;

FIG. 3C schematically depicts a cross section of the embodiment of the flushing section of FIG. 3A, along line C-C of FIG. 3A;

FIG. 3D schematically depicts an elongate element of a flushing section according to an embodiment of the invention;

FIG. 3E schematically depicts an elongate element of a flushing section according to another embodiment of the invention;

FIG. 4A schematically depicts a reproductive tract of a sow or a gilt;

FIG. 4B schematically depicts the reproductive tract of FIG. 3A, with the insertion tube according of an embryo collection device according to an embodiment of the invention inserted therein;

FIG. 4C schematically depicts the embryo collection device of FIG. 3B, wherein the flushing catheter is inserted further into the uterus;

FIG. 4D schematically depicts the embryo collection device of FIG. 3C, upon rotation of the flushing catheter with respect to the insertion tube to steer the flushing catheter into a selected uterine horn;

FIG. 4E schematically depicts the embryo collection device of FIG. 3D, wherein the flushing catheter is advanced further into the selected uterine horn, and wherein the inflatable cuff is inflated;

FIG. 5A schematically depicts bottom view of a flushing section according to an embodiment;

FIG. 5B schematically depicts an enlarged perspective view of the connector of the flushing section of FIG. 5A;

FIG. 5C schematically depicts a side perspective view of the connector of FIG. 5B;

FIG. 6A schematically depicts a perspective view of a flushing section connector according to another embodiment; and

FIG. 6B schematically depicts a side view of the flushing section connector of FIG. 6A.

Throughout the figures, the same reference numerals are used to refer to corresponding components or to components that have a corresponding function.

### DETAILED DESCRIPTION OF EMBODIMENTS

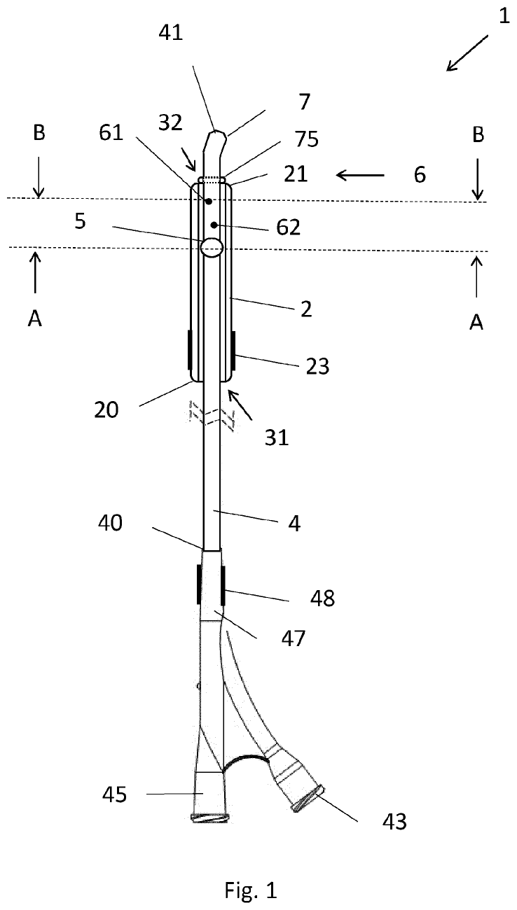

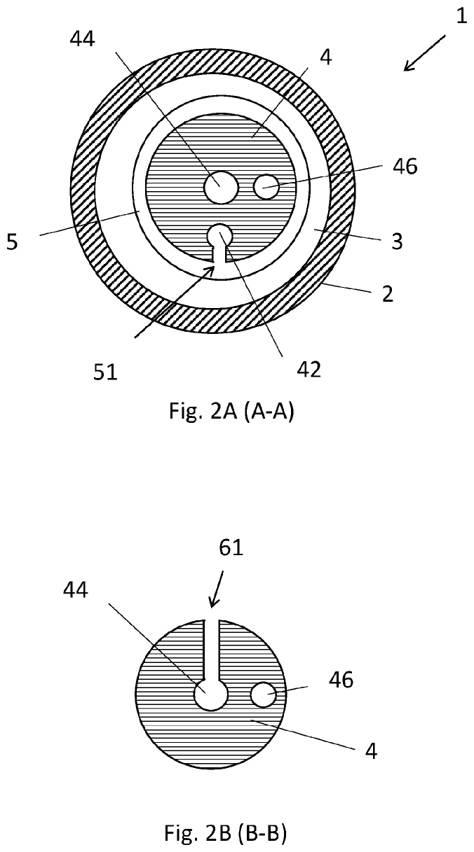

FIGS. 1, 2A and 2B schematically depict an embryo collection device 1 according to an embodiment of the invention. The embryo collection device 1 comprises an introduction probe 2 to be partially introduced into the cervix of the pig, provided with a guide lumen 3;

The embryo collection device 1 further comprises a flushing catheter 4, slidably arranged in the guide lumen 3. The flushing catheter 4 comprises a proximal end 40 and a distal end 41, an inflation lumen 42 extending from an inflation port 43 at the proximal end 40 and a flushing lumen 44 extending from an flushing port 45 at the proximal end. At the proximal end 40, a manifold 47 is provided having the inflation port 43 and the flushing port 45.

The introduction probe 2 has a proximal end 20 and a distal end 21. The introduction probe 2 is a relatively rigid, straight tube provided with a flat outer surface, having a length of at least 40 cm, in particular 50 cm.

The guide lumen 3 extends from a proximal opening 31 in the proximal end 20 up to a distal catheter opening 32 in the distal end 21. The introduction probe 2 has, towards the distal end 41, a decreasing outer diameter and a rounded end surface 21. The introduction probe 2 is relatively stiff and configured to be positioned, from the perspective of an operator, in front of the caudal opening of the cervix. The proximal end 20 comprises a grip 23. Further, the manifold 47 of the flushing catheter 4 is provided with a grip 48.

The flushing catheter 4 comprises a collar 75 having an outer diameter that is larger than a diameter of the distal catheter opening 32. Additionally or alternatively, the introduction probe 2 may comprise a holding element to hold the flushing catheter 2.

The flushing catheter 4 is configured to extend through the distal opening 32 in communication with the guide lumen 3 at the distal end 21 of the introduction probe 2 and comprises an inflatable cuff 5 fluidly connected to the inflation lumen 43 via a side port 51 to be inflated for at least partially closing the uterine horn at a proximal end thereof.

The flushing catheter 4 comprises a flushing section 6 arranged between the inflatable cuff 5 and the distal end 41 to be introduced inside the uterine horn. The flushing section 6 comprises multiple flushing openings 61, 62, 63 evenly distributed over the length of the flushing section 6 and evenly spaced around the circumference thereof. The flushing openings 61, 62, 63 are fluidly connected to the flushing lumen 44 and have a diameter of equal to a diameter of the flushing lumen 44, which is 2-6 mm, in particular 4 mm in this embodiment.

The flushing section comprises a tip element 7 arranged at the distal end 41. The tip element 41 is shaped to steer the flushing catheter 4. The flushing catheter 4 is configured to be advanced through the cranial opening of the cervix when the introduction tube 2 is inserted partially into the cervix and not through the cranial opening of the cervix.

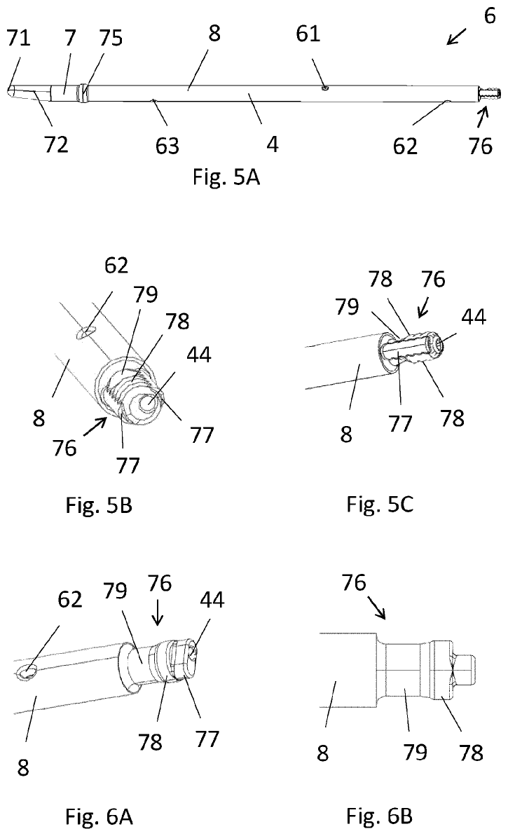

The tip element 7 and/or the flushing section 6 may be connected to the rest of the catheter via a detachable connection, such as threaded connection 74. As such, the flushing section 6 in this embodiment is detachable from the rest of the catheter that comprises the inflatable cuff 5. Instead of a threaded connection 74, other connections types may be used, such as the connectors 76 depicted in FIGS. 5A-C and 6A-B.

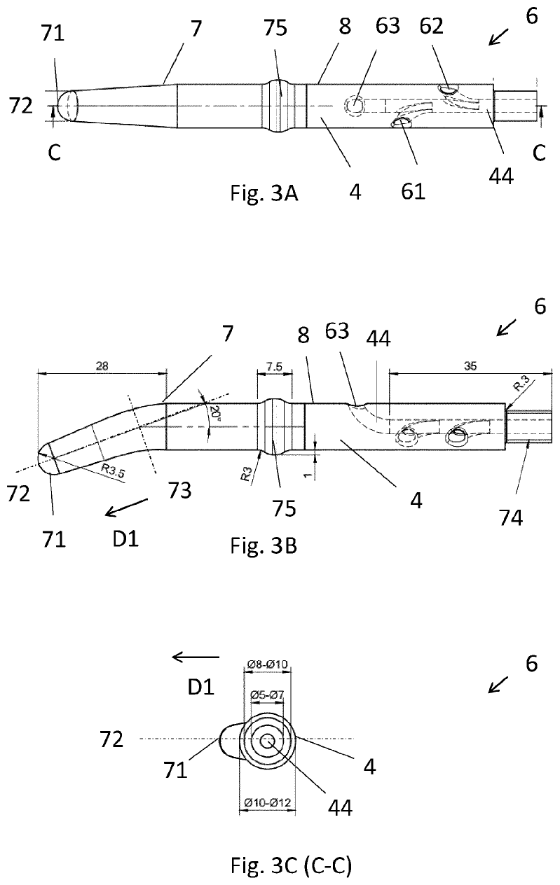

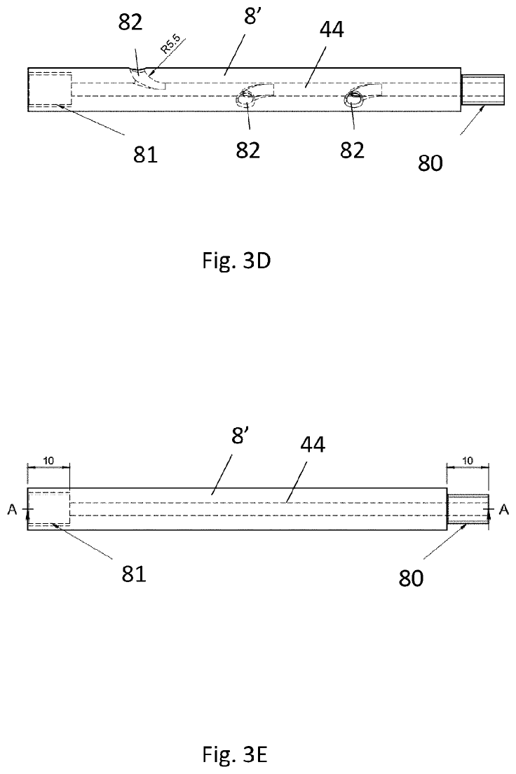

As shown in FIGS. 1, 3A, 3B and 3C, the tip element 7 is a rounded cone, in particular an oblique cone having a rounded top 71 at the distal end 41 that is bent having the cone axis 72 in a first biasing direction D1 at an angle that is less than 25°, in particular 5°-10°, for example 8°, with respect to the longitudinal axis of the flushing section 6.

The top 71 may also be biased in a second biasing direction, such that a projection of the top 71 on a cross sectional base plane 73 is not located in the centre of the base plane 73. The second biasing direction may coincide with the first biasing direction D1. The tip element 7 may be bent in the second biasing direction.

As an example only, dimensions (in mm) are shown in FIGS. 3B, 3C, 3D and 3E.

The flushing catheter 4 may be provided with additional lumina 46 (not shown in FIGS. 1, 3A-3E) fluidly connected to additional side ports (not shown), such as a collection lumen extending from an embryo collection port at the proximal end 40, for example arranged in the manifold 47, towards the flushing section, wherein the flushing section comprises a collection opening fluidly connected to the collection lumen. The collection lumen may have similar properties and dimensions as the flushing lumen 44. The flushing opening and the collection opening may be spaced at a distance from each other, in particular wherein the collection opening is spaced towards the inflatable cuff 5, and wherein the flushing opening is spaced towards the tip element 7. A similar distribution may be used when multiple collection and/or flushing openings are provided. In particular, the flushing opening may be located in the tip element such that flushing fluid can be provided near the distal end 41.

The flushing catheter 4 has a relatively high torsional stiffness to allow for rotation around a longitudinal axis such that the tip element 7 may be rotated by rotating a proximal end 40 of the catheter 4 and that a uterine horn may be selected by rotation of the tip element 7 from the proximal end 40. The flushing catheter 4 has relatively low bending stiffness for following the shape of the uterine horn and a relatively high longitudinal stiffness.

The flushing section 6 comprises an elongate element 8 that spaces the inflatable cuff 5 from the tip element 7 along the longitudinal axis, such that the inflatable cuff 5 is arranged on a fixed distance to the tip element 7 and that a relatively constant length of the uterine horn may be flushed. The flushing openings 61, 62, 63 are provided in the elongate element 8.

However, for adapting the distance between the tip element and the inflatable cuff 5, additional elongate elements 8′ may be provided, as shown in FIGS. 3D and 3E. As an example only, dimensions (in mm) are shown in FIGS. 3D and 3E. The additional elongate element 8′ may have additional openings 82 and may be provided with connections, such as threaded connections 80, 81 corresponding to the connections, e.g. threaded connections 74, of the flushing section 6, tip element 7 and/or other parts of the flushing catheter 4 for fluidly connecting the additional openings 82 to the flushing lumen 44. Other examples of connections are depicted in FIGS. 5B-C and 6A-B.

The tip element 7 is less rigid than the elongate element 8.

A total length from the top 72 at the distal end to the inflatable cuff 5 is 5-70 cm, for example 20-55 cm, in particular 30-40 cm in this embodiment. The inflatable cuff has an inflated volume of at least 10 mL in the inflated state, for example at least 50 mL.

A length of the flushing catheter 2 from the proximal end 40 up to the inflatable cuff 5 is at least 100 cm, for example at least 150 cm, in particular 200 to 350 cm. An outer diameter of the flushing catheter 2 is at least 4 mm, for example 8 or 10 mm.

The tip element 7 is autoclave-resistant for at least 15, such as 30, minutes at 121° C., and/or at least 3, such as 6, minutes at 134° C., for example at a steam pressure of 2-5 bar.

The flushing section 6 including the tip element 7 and the flushing lumen 44 have a smooth surface. The flushing catheter 2 comprises a silicone rubber and the tip element is made of a 3d-printed silicone rubber having an elastic modulus of 2.5-7.5 N/mm², such as TrueSil A50.

The tip element 7 is connected to the elongate element 8 at collar 75 via a silicone glue, such as Dow Corning 782.

The embryo collection assembly 1 may comprise an imaging system such as an ultrasound or pulsating doppler system, for imaging a position of the tip element with respect to the uterine horn.

Another embodiment of a flushing section 6 is depicted in FIG. 5A. Other properties may be similar to the flushing section 6 of FIG. 3A. The flushing sections comprises a connector 76, which is depicted in FIGS. 5B and 5C. Another possible connector 76 is depicted in FIGS. 6A and 6B. Complementary connectors may be arranged on additional elongate elements 8′ and/or on the rest of the catheter having the inflatable cuff 5.

The connector 76 comprises radial protrusions 77 that are arranged on the flushing section in a non-rotationally symmetrical way, such that rotation forces may be transferred reliably via the connector 76. The radial protrusions 77 provide the connector with a non-circular cross sectional shape, in particular an oval.

The advantage of the connector 76 of FIGS. 5A-5C and 6A-6B is that no screw thread may be necessary for attachment, allowing a wall thickness of the catheter to be relatively large causing a relatively large transversal strength of the connector 76. Additionally or alternatively, a glue, a friction lock, snap fit or other connection is used. For example, an indent 79 may be provided having a first diameter and one or more ridges 78 may be provided having a second diameter larger than the first diameter, such that the connector 76 may be held via glue, friction with the one or more ridges 78 and/or snapping around indent 79 behind a ridge 78.

The flushing openings 61, 62, 63 are fluidly connected to the flushing lumen 44 and have a diameter of equal to a diameter of the flushing lumen 44, which is 2.5 mm in this embodiment.

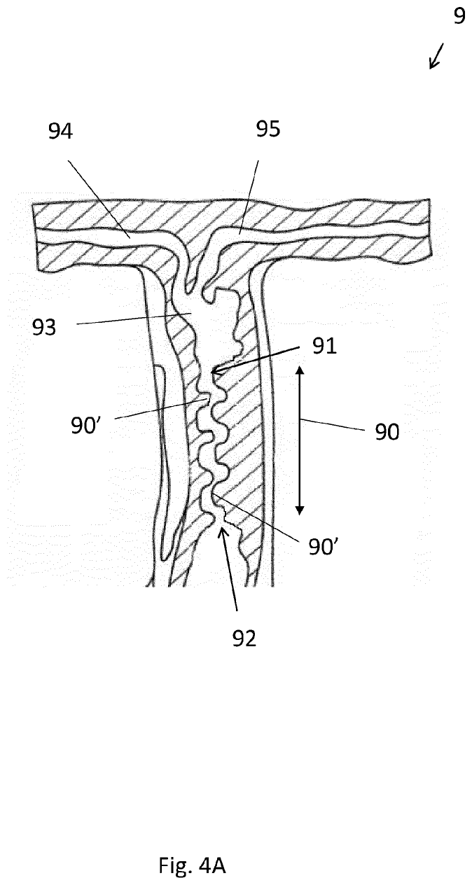

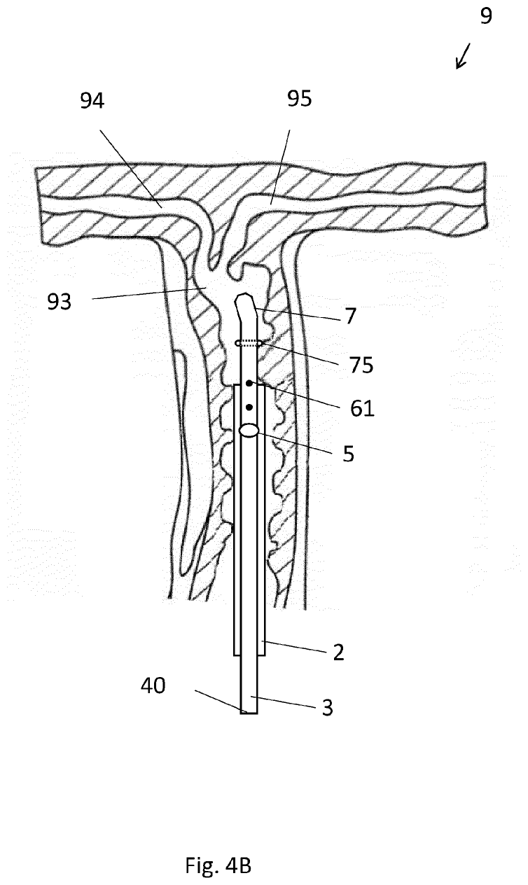

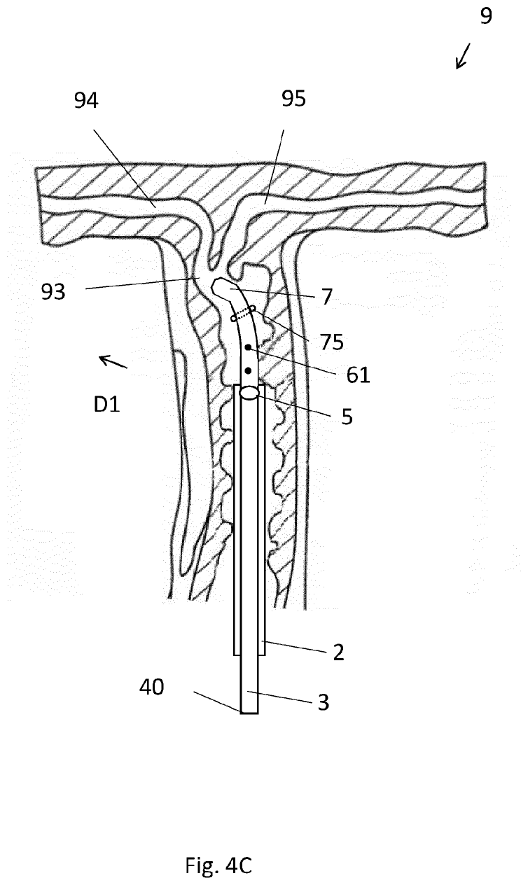

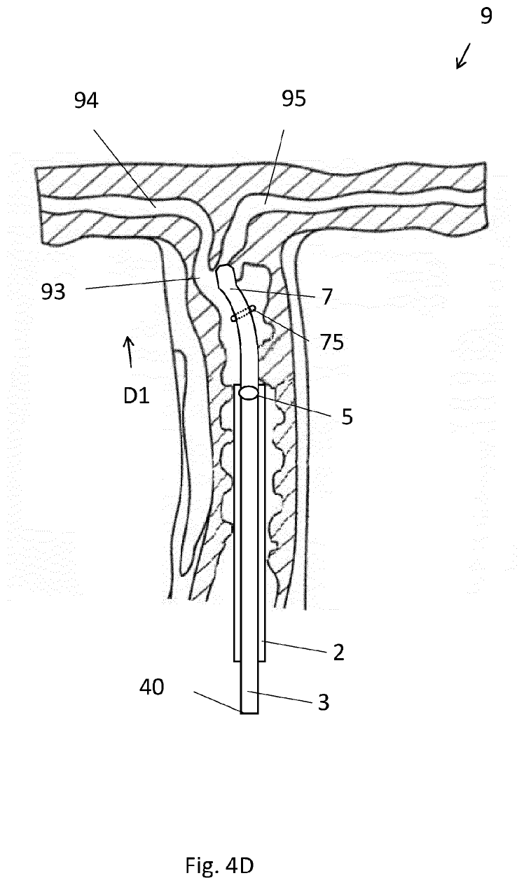

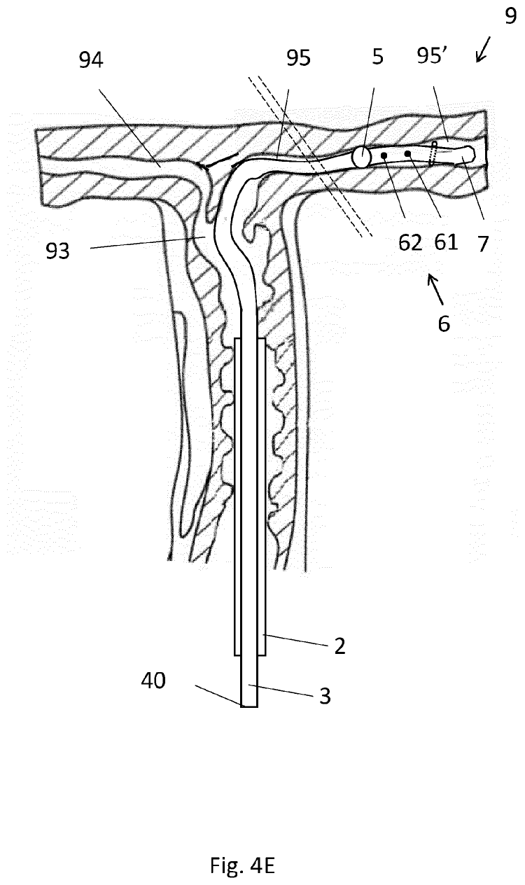

FIGS. 4A-4E schematically show the use of an embryo collection device according to an embodiment of the invention for collecting embryos in the uterine horn of a sow or a gilt.

The reproductive tract 9 of a sow or a gilt comprises a cervical canal 90 extending between the relatively narrow caudal opening 92 and cranial opening 91. The cervical canal 90 comprises cervical folds 90′.

Using the embryo collection device 1, the introduction probe 2 may be inserted partially into the cervical canal 90, for example through the caudal opening 92 but not through the cranial opening 91 as an operator can feel the position of the introduction probe 2 with respect to the cervical tissue. The embryo collection device 1, is, via the introduction probe 2, first inserted for approximately 50 cm into the reproductive tract.

The cervix may be passed in a relatively short time, for example within 20-30 seconds. In particular, the introduction tube 2 may be positioned in front of the cranial opening 91 within 20-30 seconds.

Upon insertion of the introduction probe 2 in the cervical canal 90, the flushing catheter 2 may be advanced further for at least 50 cm to extend out of the introduction probe 2 through the distal catheter opening 32 into the reproductive tract passing the cervix and uterine body as shown in FIGS. 4B and 4C, without being supported by a guide wire and without being supported by the introduction tube 2 through the entire cervix.

Due to the longitudinal stiffness of the tip element 7, a position thereof with respect to the uterine horns 94, 95 may be determined haptically by an operator. Alternatively, a position may be determined using an imaging system.

Then, by orienting the tip element 7 by rotating distal end 40 around its longitudinal axis, a position of the rounded cone with respect to uterine horns 94, 95 may be selected, as depicted in FIG. 4D. This way, the first biasing direction D1 and/or second biasing direction defined by the tip element 7 may rotated to coincide with an opening of one of the uterine horns 94, 95.

The tip element 7 may be advanced into a selected uterine horn 95 and, by means of the biasing direction D1, is configured to steer the flushing catheter 3 into and through the selected uterine horn 95. The flushing catheter 4 may be positioned to perform flushing at a desired depth in the uterine horn, for example deep in the uterine horn

As shown in FIG. 4E, upon advancing the flushing catheter 3 into the selected uterine horn 95 towards a desired depth, the inflatable cuff 5 may be inflated via the inflation lumen to at least partially close a part 95′ of the uterine horn, after which flushing of the at least partially closed-off part 95′ may be performed by providing an amount of flushing fluid through a flushing port, flushing lumen and the flushing openings 61, 62 of the flushing section 6. A flushing volume of at least 0,25 L is used, in particular 0,5 L.

Due to the deep positioning in the uterine horn 95′ and the inflation of the inflatable cuff 5 at a desired depth, at least 40% of the flushing volume of flushing fluid is recovered. In particular 50-70% of the flushing volume is recovered.

Flushing may be performed 3-9 days after observing a standing reflex of the sow, in particular on day 5-7. The presence and/or properties of the embryos may be determined, in particular blastocysts, e.g. by imaging the ovaries of the pig using an imaging system. Ovulation may for example be detected by imaging the ovaries with ultrasound.

The embryo collection device may at least be partially sterilised in an autoclave.

1. An embryo collection device for collecting embryos in the uterine horn of a pig, comprising:
an introduction probe to be partially introduced into the cervix of the pig, provided with a guide lumen; and
a flushing catheter, slidably arranged in the guide lumen, comprising:
a proximal end and a distal end, an inflation lumen extending from an inflation port at the proximal end and a flushing lumen extending from a flushing port at the proximal end;
an inflatable cuff fluidly connected to the inflation lumen to be inflated for at least partially closing the uterine horn towards a proximal end thereof; and
a flushing section arranged between the inflatable cuff and the distal end to be introduced inside the uterine horn, comprising a flushing opening fluidly connected to the flushing lumen,
wherein the flushing section comprises a tip element arranged at the distal end, and wherein the tip element is shaped to steer the flushing catheter.

2. The embryo collection device according to claim 1, wherein the tip element is a rounded cone.

3. The embryo collection device according to claim 2, wherein the tip element is a bent rounded cone.

4. The embryo collection device according claim 1, wherein the flushing section comprises an elongate element that spaces the inflatable cuff from the tip element in a longitudinal direction.

5. The embryo collection device according to claim 4, wherein the flushing opening is arranged in the elongate element and/or in the tip element.

6. The embryo collection device according to claim 4, wherein the tip element is less rigid than the elongate element.

7. The embryo collection device according to claim 1, wherein a length of the flushing catheter from the proximal end up to the inflatable cuff is at least 100 cm.

8. The embryo collection device according to claim 1, wherein the tip element is autoclave-resistant.

9. The embryo collection device according to claim 1, wherein the flushing catheter comprises a collection lumen extending from an embryo collection port at the proximal end towards the flushing section, and wherein the flushing section comprises a collection opening fluidly connected to the collection lumen.

10. The embryo collection device according to claim 1, wherein the introduction probe is straight, and/or wherein the introduction probe has a flat outer surface.

11. An embryo collection assembly, comprising:
the embryo collection device according to claim 1;
a flushing pump fluidly connectable to the flushing lumen via one of the at least two ports for pumping flushing fluid through the flushing opening;
an inflation pump fluidly connectable to the inflation lumen via one of the at least two ports for inflating the inflatable cuff; and
an embryo collection container, fluidly connectable to one of the at least two lumens for collecting embryos flushed out of the uterine horn.

12. The embryo collection assembly according to claim 11, comprising an imaging system for imaging a position of the tip element with respect to the uterine horn.

13. A method of using the embryo collection device according to claim 1, comprising the step of:
collecting embryos in the uterine horn of a sow or a gilt with the embryo collection device.

14. The method according to claim 13, wherein the introduction probe is introduced into the cervix but not advanced through the entire cervix, for example introduced up to ⅔ of the length of the cervix,

15. The method according to claim 13, wherein the tip element is advanced up to at least 50% of the uterine horn, for example up to the tip of the uterine horn.

16. The method according to claim 13, performed 3-9 days after observing a standing reflex of the sow, in particular on day 5-7.

17. The method according to claim 13, comprising the step of recovering 50-70% of the volume of flushing fluid.

18. A method for flushing a reproductive tract using the embryo collection device of claim 1, comprising the steps of:
moving the introduction probe having the guide lumen partially into the reproductive tract in a movement direction;
moving the flushing catheter arranged in the guide lumen to slide within the guide lumen in the movement direction;
rotating the flushing catheter with respect to the introduction probe to steer the flushing catheter with the tip element thereof in a direction angled to the movement direction;
further moving the flushing catheter at a proximal end in the movement direction;
inflating the inflatable cuff;
providing flushing fluid into the flushing lumen; and
collecting flushing fluid from the flushing lumen and/or from a collection lumen.

19. The embryo collection device according claim 4, wherein a length from the distal end to the inflatable cuff is 5-70 cm.

20. The embryo collection device according claim 4, wherein a length from the distal end to the inflatable cuff is 30-40 cm.
